(19) United States
(12) Patent Application Publication
(10) Pub. No.: US 2014/0052223 A1
(43) Pub. Date: Feb. 20, 2014

(54) SYSTEM AND METHOD FOR CONTROLLING A TANNING BED

(71) Applicant: ZOOM TAN, INC., Naples, FL (US)

(72) Inventor: Tony TOEPFER, Naples, FL (US)

(73) Assignee: ZOOM TAN, INC., Naples, FL (US)

(21) Appl. No.: 13/839,052

(22) Filed: Mar. 15, 2013

Related U.S. Application Data
(60) Provisional application No. 61/684,500, filed on Aug. 17, 2012.

(51) Int. Cl. A61N 5/06

(52) U.S. Cl. CPC A61N 5/0614
USPC 607/91

(57) ABSTRACT

Described in this application is a novel method for providing tanning services that includes the steps of connecting an output port of a portable device to an input port of a tanning bed, transmitting a signal from the portable device to the tanning bed through the connection and enabling the tanning bed in response to the signal.

### CROSS-REFERENCE TO RELATED APPLICATION

This application claims priority to U.S. Provisional Patent Application No. 61/684,500, filed Aug. 17, 2012, which is incorporated herein by reference.

### BACKGROUND

The present invention relates to a system and method for providing tanning services. More particularly, the present invention relates to a system and method for using a software program to remotely reserve tanning services and electronically interface with a tanning bed to provide tanning services.

Tanning bed salons are popular destinations for individuals seeking to tan year round. These salons typically include a number of beds that individual users reserve for a period of time, allowing the user to privately tan for a fixed period of time. Individuals wishing to reserve time on a tanning bed contact a clerk or other central customer-service representative who monitors and manages the various beds. The customer-service representative assigns a bed to an individual and allots a specific amount of time to that user.

During the user's allotted time, the tanning bed is operational, allowing the user to use the tanning bed for as long as they wish. Once the user's allotted time has expired, the tanning bed is no longer operational.

It is desired to provide a tanning system whereby individuals may use and control tanning beds by means of remote access, such as a smart phone or other portable communication device. It is further desired to provide a system whereby control of tanning beds may be performed without requiring an employee to provide and monitor bed access. It is further desired to provide a system whereby control of the tanning beds may be automated.

### SUMMARY

Described in this application is a novel method for providing tanning services. The method comprises the steps of connecting an output port of a portable device to an input port of a tanning bed, transmitting a signal from the portable device to the tanning bed through the connection, and enabling the tanning bed in response to the signal.

According to various other arrangements, the additional step of severing the connection and disabling the tanning bed when the connection is severed are provided.

In other embodiments, the portable device may be a smartphone and a cable may be provided that is adapted to engage the input port and the output port. The output port may be an audio jack. Alternatively, the output port may be a data jack.

Also described is a novel system for providing tanning services. The system includes a portable device having a user interface and an output port capable of transmitting a signal and a tanning bed for providing tanning services and having an input port for receiving the signal. The portable device is configured to transmit the signal from the portable device to the tanning bed. When received, the tanning bed is enabled, allowing a user to use the bed for tanning.

According to various other arrangements, the tanning bed is disabled when the signal is terminated, or the tanning bed may be disabled after some duration.

### BRIEF DESCRIPTION OF DRAWINGS

Reference is made to the accompanying drawings in which particular embodiments of the invention are illustrated as described in more detail in the description below, in which:

FIG. 1 is a schematic view of a tanning bed system according to the invention;

FIG. 2 is a schematic diagram of electronic connections between various components that may be used in embodiments of the disclosed system.

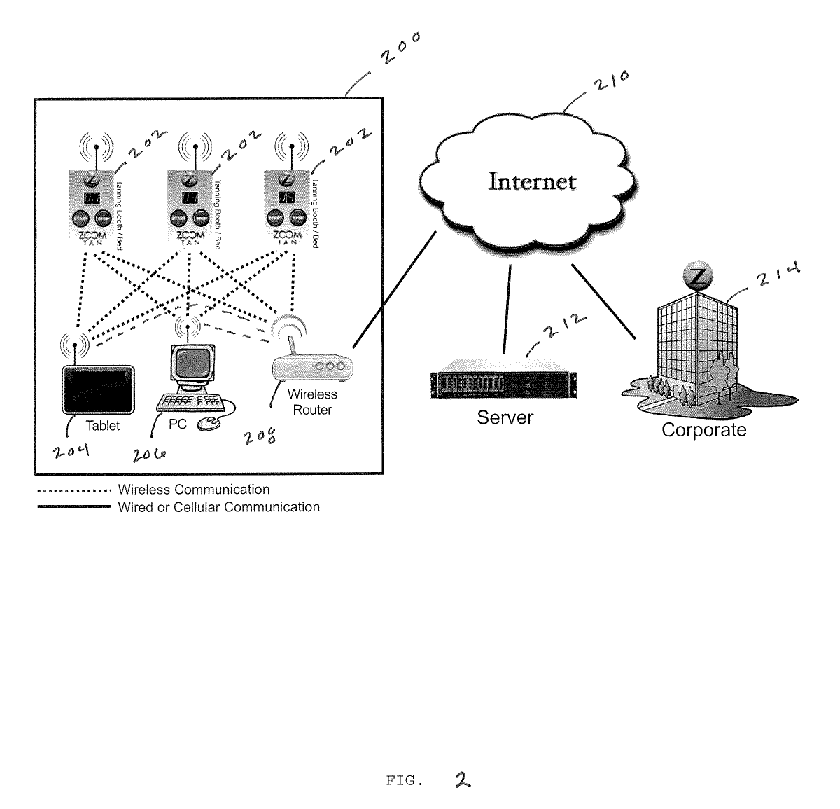

FIG. 3 is an embodiment of a user interface for a tanning bed.

### DETAILED DESCRIPTION

Presently disclosed is a method for coordinating, managing, and controlling tanning bed usage. Also disclosed is a software application for reserving, purchasing, and monitoring tanning bed access. Also disclosed is a apparatus and method for communicating between a portable communication device; such as a smart phone, tablet, laptop, or other device; and a tanning bed.

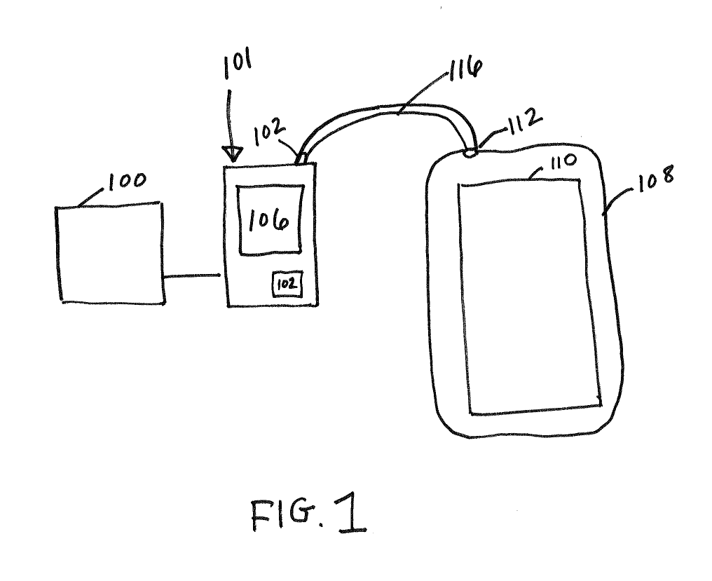

In an embodiment, as illustrated in FIG. 1, a tanning bed 100 is provided that includes a timer 102, input port 104 and a digital display 106. Also provided is a phone 108 with an interface screen 110, output port 112, and wireless connection. A cable 116 may connect the output port 112 of the phone 108 to the input port 104 of the tanning bed 100.

In one embodiment, a software application is provided. This software application may be a web application, a smartphone application, a stand-alone application, or other type of software product capable of communicating with a server. This communication may be wired or wireless, pass through the internet or a local network, and may be a Bluetooth, 4G, wi-fi, or other type of network connection.

An individual user wishing to reserve a tanning bed at a salon may input into the software application a request to reserve and purchase a period of time at a tanning bed in a specific tanning salon. This information is communicated by the software application to a central server. The information may include the requested time slot (date, time, and duration), the user's personal information and tanning salon location. The central server may check the availability of the tanning salon for the requested time slot and return a signal to the software application indicating whether the requested time slot is available. If not available, the user may be prompted to enter another time slot, or may be provided with a number of available time slots or locations.

Once a time slot has been indicated to the software application as available then the individual may next be prompted with an option to reserve that time slot, or the time slot may be automatically reserved. This automatic reservation may be temporary until the user purchases the time slot or may be permanent or revocable. This reservation is stored centrally by the central server and may exclude other users from reserving the requested time slot at the selected location.

The user will next be prompted to purchase the selected time slot through the software application through a variety of payment methods, including credit card, debit card, fund transfer, electronic check, debiting or charging account with the tanning salon. The central server may then transmit a reservation code to the software application completing the reservation and allowing the user to access a tanning bed at the salon during the requested time slot. The central server may also transmit the reservation to a local tanning salon server.

When the user arrives at the tanning salon, the user may check-in with a tanning salon server or individual. This check-in may be performed by either electronic or physical observation, confirming the user either by visual observation or by recognizing the user by means of the software application residing on a user's smart phone. The tanning salon server may determine which tanning beds are available and block off the reserved time slot for a selected tanning bed. Alternatively, an individual tanning bed may have already been blocked off for the user. Each tanning bed may include a unique address, such as an internet protocol (IP) address, including IPv4. The unique address of the selected tanning bed may next be communicated to the software application through a variety of methods including internet, Bluetooth or other wired or wireless communication.

In order to access the tanning bed, a user may be required to electronically interface with the tanning bed via the smart phone application. This electronic interface may be performed by means of a wired or wireless connection. The electronic interface includes an encoded signal from the smart phone application to the tanning bed that is equal or related to the reservation code provided by the central server. The tanning bed may transmit this code to the tanning salon server or central server to verify the identity of the user. Alternatively, the tanning bed may receive the reservation code when the reservation is confirmed and may compare this code to the code provided by the user. Once the user has been verified as the individual who reserved the tanning bed the tanning bed may become operational, allowing the individual to use the tanning bed.

In one embodiment, a wire may be provided between an output port on the phone, such as the audio output, and an input port on the tanning bed adapted to receive a wire from the smart phone. The input port may be adapted to receive, for example, a TRRS connector, mini- or micro-USB, or other electrical connector capable of conveying a signal from the smart phone to the tanning bed.

In another embodiment, the input port of the tanning bed may be a Bluetooth receiver that forms a local network with the user's smart phone, allowing signals to be securely and wirelessly transmitted between the smart phone and the tanning bed.

In yet another embodiment, the input port of the tanning bed may be a wired or wireless connection, such as an Ethernet or Wi-Fi connection, to the tanning salon server or to the central server. The smart phone application may communicate with the tanning bed through the internet connection and the server.

In various embodiments, the tanning bed is only reserved for a selected duration that may be measured in minutes, hours or other increments. The amount of time the tanning bed is active and providing light suitable for tanning may be controlled by a separate timer, such as the user's smart phone, the tanning salon server, the central server or the tanning bed itself.

The signal communicated from the smart phone to the tanning bed may include embedded information, such as the reserved duration, and the tanning bed may include a decoder for retrieving the embedded information from the signal. In an embodiment, the tanning bed includes a timer or is in communication with an external timer. Once the decoder retrieves the embedded information, such as a tanning duration, the timer may be set to allow the tanning bed to operate for the requested duration.

In other embodiments, the smart phone application may include a timer. Once the smart phone has interfaced with the tanning bed and transmitted the signal to the tanning bed the timer may begin a countdown (or countup) until the reserved duration has been exhausted. After the reserved duration has been exhausted the smart phone application may transmit a termination signal to the tanning bed indicating that the reserved time has been exhausted.

In another embodiment, the smart phone may continuously transmit a signal to the tanning bed and the tanning bed may remain in operation while the signal is being received. Once the signal has ceased, either because the timer has been exhausted or because the wired or wireless connection between the smart phone and tanning bed has been interrupted or terminated, the tanning bed will cease to operate.

In yet another embodiment, the tanning bed may include a detector for sensing when the smart phone has been connected to the tanning bed. In one arrangement, the connection between the smart phone and tanning bed is by means of a wire from the smart phone audio output to the input port on the tanning bed. The tanning bed may include an electronic or physical sensor for detecting when the bed has been connected to the phone. When the wire is disconnected from the bed or phone operation of the tanning bed may cease.

In yet another embodiment, a timer may be provided remote from the tanning bed, such as at the central server or tanning salon server. When the tanning bed receives a key signal from the smart phone application the tanning bed may communicate, either wirelessly or wired, with the remote timer. The timer may return an activation signal to the tanning bed, indicating that the bed should become operational for the user. Once the timer has been exhausted a termination signal may be sent to the tanning bed, indicating that the bed should cease operation.

While the tanning bed may only be operational for the reserved time duration, it is contemplated that the tanning bed, central or tanning salon server, or smart phone application may include means for alerting the user that the user's time is about to expire and prompt the user to reserve additional time by extending the time slot, if available. This alert may include audio and/or visual signals at either or both of the tanning bed and the smart phone application.

It is further contemplated that the smart phone or tanning bed may be able to set individual tanning sessions within the reserved time slot and start and stop the individual tanning session at any time. An individual tanning session commences when the lights in the tanning bed or booth are engaged, providing the user with light of a level and wavelength suitable for tanning. A separate timer may be engaged, either on the bed, phone, or either server that measures the duration of the individual tanning session. The timer may show remaining time which will count down in minute or second intervals until zero is reached, at which point the individual tanning session will terminate.

In embodiments, an arming function is provided in the tanning bed or server that detects when the tanning bed is in use for an individual tanning session. If an individual tanning session is not commenced within a defined time period, such as 15 minutes, of the user connecting to the tanning bed and transmitting a signal then the tanning bed may be shut down. Alternatively, any individual session may be interrupted and restarted by the user, with the use of a stop or pause button so long as the user has time remaining in their individual tanning session.

In some embodiments, a proximity detection function may also be provided, either separately or in connection with the arming function. The proximity detection function operates to determine if a user is sufficiently near a tanning bed to be permitted to arm and/or activate the tanning bed. The proximity detection function may be particularly useful when the user's portable communication device communicates wirelessly with the tanning bed. The tanning salon may wish to prohibit a user from arming or activating a bed for another user, or from arming or activating multiple beds at the same time. Due to the tendency for users to remain in close proximity to their smart phone, determining whether the smart phone is sufficiently close to the tanning bed increases the probability that the correct user is operating that particular tanning bed. The proximity detection function may rely upon the strength of a a wireless signal, or may rely upon geolocation techniques to determine the location of the phone relative to the tanning bed. In embodiments using wired communication between the user's smart phone and the tanning bed controller, the proximity detection function may detect the presence or absence of the connecting cable. In each case, the proximity detection function operates to prevent a tanning bed from being armed or activated by a distant user, or to disable a tanning bed if the user leaves the proximity of the tanning bed during the tanning session.

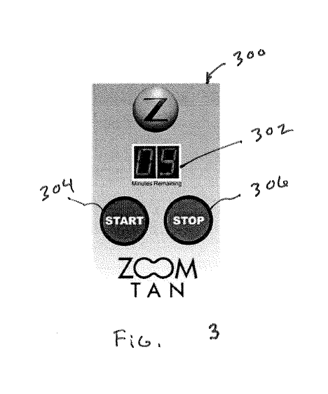

Finally, a user interface for the tanning bed is provided. The user interface may be, for example, a printed self adhesive overlay with integrated membrane switches and one or more 7-segment LED displays behind a transparent screen that allows the user to set, start, and end individual tanning sessions. The user interface may include start and stop buttons for beginning, ending, and restarting an individual tanning session. The user interface may also include buttons to increase or decrease the minutes remaining in an individual tanning session. One example of a user interface is illustrated in FIG. 3. The user interface 300 includes a timer display 302, a start button 304, and a stop button 306.

According to various other embodiments, the user interface is a wirelessly controlled relay that communicates with the tanning salon server and a user's smart phone to manage and control individual tanning sessions. The user interface may also communicate with the internet through a wireless router to a remote server, such as the central server, and receive information therefrom.

According to an embodiment, the user interface for the tanning bed is a tablet computer configured to communicate with a user's portable electronic device and with the tanning bed. The tablet computer may implement a wireless controlled relay as described above to communicate with a tanning salon server and the user's smart phone to manage and control individual tanning sessions. In one embodiment, the user of a given tanning bed is known due to the communication between the user's smart phone and the tanning bed system. The user interface may be controlled to display a customized greeting to the user. In addition, the system may display advertising or promotional material tailored to the user. In embodiments, the system is configured to access a user's account, such as may be stored in a corporate database, and use this information, including the user's demographic information and prior tanning or purchase history, to select advertisements to be displayed for the specific user.

According to yet other embodiments, the time length of an individual session may be set, started, stopped, and reset by other than the user interface. This may include portable communication devices, the central server, tanning salon server, desktop PC, office staff, or other electronic communication devices.

According to yet another embodiment, a software application may be provided on a user's smart phone or other similar portable communication device. The user may reserve a duration for an individual tanning session. Upon arrival at the tanning salon, the user may electronically interface with the tanning bed, through either a wired or wireless connection, and the individual tanning session will commence.

The individual tanning session includes a duration, for example ten minutes. During this duration, the software application may continuously transmit a signal to the tanning bed. Once the duration has concluded, the software application may terminate the signal and the individual tanning bed may be ended.

Further, either the software application or the tanning bed may include a control for the tanning bed. The control may include a stop button in addition to arrows providing for an increased or decreased duration. By engaging each of these buttons the user may alternatively stop the tanning session, increase the duration of the tanning session or decrease the duration of the tanning session. Other controls are also contemplated, for example the software application may allow a user to transmit music from another application through speakers in the tanning bed allowing the user to listen to music while tanning.

In some embodiments, the system may determine a maximum tanning time for a given user based on a user's tanning history. For example, a user who has not used the tanning salon for an extended period may be limited to a minimum tanning duration so as to prevent overexposure and possible injury to the user. In contrast, a user who tans frequency may be permitted to gradually increase the duration of their tanning sessions. Through the communication between a user's portable communication device and the tanning bed controller, the system is able to identify the user and may access that user's tanning history. If a user desires to tan longer than the system determines is appropriate, the user may be required to consult with a tanning salon employee who may provide an assessment of the user's request. In this manner, the system may provide guidance on proper tanning procedures and enforce limitations to prevent misuse of the tanning beds.

FIG. 3 shows one embodiment of the wireless tanning booth/bed controller. The tanning salon 300 includes a plurality of tanning beds each having a tanning bed controller 202 that may include a wirelessly controlled relay. One or more of a wireless router 208, personal computer 206, or portable communication device 204 such as a tablet computer are also provided, each in wireless communication with some or all of the tanning booths or beds. One or more of the portable communication device 204, the personal computer 204 and wireless router 208 may be in communication with each other, and may be in communication with external computing resources such as corporate database 214 or server 212. The portable communication device 204, the personal computer 204 and/or the wireless router 208 may communicate with other computing resources using a wired, wireless or cellular communication, and may access the external resources through the Internet.

In the specification and claims, reference will be made to a number of terms that have the following meanings. The singular forms “a”, “an” and “the” include plural referents unless the context clearly dictates otherwise. Approximating language, as used herein throughout the specification and claims, may be applied to modify any quantitative representation that could permissibly vary without resulting in a change in the basic function to which it is related. Accordingly, a value modified by a term such as “about” is not to be limited to the precise value specified. In some instances, the approximating language may correspond to the precision of an instrument for measuring the value. Similarly, “free” may be used in combination with a term, and may include an insubstantial number, or trace amounts, while still being considered free of the modified term. Moreover, unless specifically stated otherwise, any use of the terms “first,” “second,” etc., do not denote any order or importance, but rather the terms “first,” “second,” etc., are used to distinguish one element from another.

As used herein, the terms “may” and “may be” indicate a possibility of an occurrence within a set of circumstances; a possession of a specified property, characteristic or function; and/or qualify another verb by expressing one or more of an ability, capability, or possibility associated with the qualified verb. Accordingly, usage of “may” and “may be” indicates that a modified term is apparently appropriate, capable, or suitable for an indicated capacity, function, or usage, while taking into account that in some circumstances the modified term may sometimes not be appropriate, capable, or suitable. For example, in some circumstances an event or capacity can be expected, while in other circumstances the event or capacity cannot occur—this distinction is captured by the terms “may” and “may be.”

This written description uses examples to disclose the invention, including the best mode, and also to enable one of ordinary skill in the art to practice the invention, including making and using any devices or systems and performing any incorporated methods. The patentable scope of the invention is defined by the claims, and may include other examples that occur to one of ordinary skill in the art. Such other examples are intended to be within the scope of the claims if they have structural elements that do not different from the literal language of the claims, or if they include equivalent structural elements with insubstantial differences from the literal language of the claims.

What is claimed is:

1. A method for providing tanning services comprising the steps of:
connecting an output port of a portable device to an input port of a tanning bed;
transmitting a signal from said portable device to said tanning bed through said connection; and
enabling said tanning bed in response to the signal.

2. The method of claim 1 further comprising the steps of severing said connection and disabling said tanning bed when said connection is severed.

3. The method of claim 1 wherein said portable device comprises a smartphone.

4. The method of claim 2 further comprising providing a cable adapted to engage said input port and said output port.

5. The method of claim 4 wherein said output port comprises an audio jack.

6. The method of claim 4 wherein said output port comprises a data jack.

7. A system for providing tanning services comprising:
a portable device including a user interface and an output port capable of transmitting a signal; and
a tanning bed for providing tanning services, the tanning bed including an input port for receiving said signal;
wherein said portable device is configured to transmit said signal from said portable device to said tanning bed to enable use of said tanning bed when said signal is received.

8. The system of claim 7 wherein said use is disabled when said signal is terminated.

9. The system of claim 8 wherein said use is disabled after a duration.

10. The system of claim 9 further comprising a timer for monitoring said duration.

11. The system of claim 10 wherein said timer controls use of said tanning bed.

12. The system of claim 11 wherein said timer is located on said tanning bed.

13. The system of claim 12 wherein said signal initiates said timer.

14. The system of claim 11 wherein said timer is located on said portable device.

15. The system of claim 14 wherein said signal is transmitted during said duration.

16. The system of claim 15 wherein said portable device is a smart phone.

17. The system of claim 16 further comprising a cable connecting said smart phone to said tanning bed.

18. The system of claim 17 wherein said signal is adapted to be transmitted through an audio port on said smart phone.

19. The system of claim 16 wherein said smart phone transmits said signal wirelessly to said tanning bed.

20. An apparatus for providing tanning services, the apparatus comprising:
a tanning bed having a timer and an input port;
a portable device including a user interface and an output port; and
wherein a signal is conveyed between said portable device and said tanning bed.
